(19) United States
(12) Patent Application Publication
(10) Pub. No.: US 2024/0206644 A1
(43) Pub. Date: Jun. 27, 2024

(54) APPARATUS AND METHODS FOR INFANT AND MEDICAL DEVICE SUPPORT

(71) Applicant: The Children's Hospital of Philadelphia, Philadelphia, PA (US)

(72) Inventor: Amy G. Kelly, Philadelphia, PA (US)

(73) Assignee: The Children's Hospital of Philadelphia, Philadelphia, PA (US)

(21) Appl. No.: 17/910,168

(22) PCT Filed: Mar. 9, 2021
(86) PCT No.: PCT/US2021/021446

§ 371 Date: Dec. 20, 2022

Related U.S. Application Data
(60) Provisional application No. 62/987,682, filed on Mar. 10, 2020.

(51) Int. Cl. A47D 13/02; A47D 15/00; A61B 50/20

(52) U.S. Cl. CPC A47D 13/02; A47D 15/006; A61B 50/20; A61B 2503/04

(57) ABSTRACT

The present disclosure relates to apparatus and methods for safely and securely holding an infant and medical equipment proximal to the infant. In particular embodiments, an apparatus may comprise an infant support device configured to support an infant proximal to a user. Exemplary embodiments may also comprise a medical equipment support device configured to support medical equipment proximal to the infant.

### CROSS-REFERENCE TO RELATED APPLICATIONS

This application claims priority to U.S. Provisional Patent Application Ser. No. 62/987,682 filed Mar. 10, 2020, the entire contents of which are incorporated herein by reference.

### BACKGROUND INFORMATION

Medically fragile infants, including those in a Newborn/Infant Intensive Care Unit (N/IICU), provide significant challenges to caregivers or parents who wish to move the infant from a bed. For example, such infants are often connected to various forms of medical equipment, including respiratory equipment, ventilator tubing, intravenous (IV) tubing, feeding tubes and monitor wiring. Moving the infant from a bed while coupled to such equipment can be a cumbersome and difficult experience that requires the supervision of medical support staff due to the risk of the unplanned extubation, accidental uncoupling of monitor wiring, infant falling, etc. In some examples, it can take several (8-12) minutes, and up to three healthcare providers (e.g. nursing and respiratory personnel) to transfer the infant from the bed. The process can be intimidating enough to some parents that they wait months before they feel comfortable enough hold their infants.

Despite these challenges, there are not consistent standards or practices for how to remove and support medically fragile infants to allow parents to hold the infant. For example, in some cases, towels may be placed onto the parent and the infant is then carefully placed on the parent and towels. The medical equipment coupled to the infant (e.g. tubes and wires) may then be secured with tape. The tape may not sufficiently support the medical equipment and can present challenges when trying to remove the tape.

A survey of NIICU providers revealed the following percentages identified primary issues relating to moving a medically fragile infant from a bed: securing connected devices 75%; maintaining correct position of baby 16%; parent caregiver immobility 6%; modesty of parent/caregiver 2%; arm stress of parent/caregiver 2%.

Despite the difficulties of moving a medically fragile infant from a bed, there are significant benefits to doing so to allow skin-to-skin contact between the infant and a parent (also referred to a “Kangaroo care”). Such benefits include improved overall outcomes for infants including decreased hospital stays and decreased infection rates. Skin-to-skin contact promotes developmentally appropriate interactions between the infant and caregiver. In a recent study, infants were found to have larger cerebral volumes, decreased motor functional deficits and increased autonomic functioning when comparing those who have been held Skin to skin vs those who have not. See e.g. Feldman et al., Maternal-Preterm Skin-to-Skin Contact Enhances Child Physiologic Organization and Cognitive Control Across the First 10 Years of Life, BIOL PSYCHIATRY 2014; 75:56-64; and Charpak N, Tessier R, Ruiz J G, et al. Twenty-year Follow-up of Kangaroo Mother Care Versus Traditional Care. Pediatrics. 2017; 139(1):e20162063. In addition, skin-to-skin contact provides increased bonding, promotes autonomy, increased maternal breast milk production and decreased prevalence of postpartum depression. Other benefits include long term decrease in infant ventilator settings, increased feeding readiness, improved sleep cycles with longer periods of uninterrupted sleep and increased observed quiet awake periods, as well as overall decreased need for sedation.

Despite the benefits of skin-to-skin contact, NIICUs remain inconsistent with this practice. According to the March of Dimes Family Support program, about 8% of NIICU staff members report skin to skin contact as routine. Of that 8%, 73% of parents with extubated infants were offered to do skin to skin vs 45% of parents with intubated patients. Decrease of implementation of the practice is related, but not limited to, a lack of initiation from nurses or parents, fear of compromising patient safety during patient transfer, heavy nursing assignments with insufficient staffing, lack of a structured skin-to-skin policy, and decreased parent education.

As discussed in further detail below, exemplary embodiments of the present disclosure address shortcomings of existing practices and provide notable benefits in comparison to such practices.

### SUMMARY

Exemplary embodiments of the present disclosure comprise devices and methods for addressing several issues, including those stated in the background information section. For instance, exemplary embodiments provide apparatus and methods to safely and securely transfer an infant from a bed to a parent or caregiver. Exemplary embodiments of the present disclosure also provide for support of medical equipment associated with care of infants, including for example, those in NIICU treatment facilities.

In addition, exemplary embodiments of the present disclosure provide significant benefits to the user. For example, exemplary embodiments provide for simplicity of transferring an infant to the person holding the infant, as well as accounting for user modesty. Exemplary support apparatus as disclosed herein can also provide multiple access points and humanize the experience for the parent. Furthermore, exemplary embodiments can be configured to fit a range of users, both infants and adults and provide for emergency removal if needed. In addition, support apparatus as disclosed herein can be made from materials that are easily laundered.

Exemplary embodiments of the present disclosure include a support apparatus comprising an infant support device configured to support an infant proximal to a user, and a medical equipment support device configured to support medical equipment proximal to the infant. In certain embodiments the infant support device comprises overlapping panels. In particular embodiments the infant support device comprises a cover configured to be coupled to a chair. In some embodiments the infant support device comprises a neck pad, which may be heated in specific examples.

In certain embodiments the infant support device comprises an adjustable neck strap and/or an adjustable waist strap. In particular embodiments the infant support device comprises a shirt configuration. In some embodiments the medical equipment support device comprises one or more holes in the shirt configuration. In specific examples the infant support device comprises an upper portion and a lower flap portion. In certain embodiments the lower flap portion can be coupled to the upper portion, including via buckles or via hooks that engage holes in the upper portion.

In particular embodiments the infant support device comprises a holster configuration. In some embodiments the infant support device comprises a sling, which may comprise a stretchable fabric. Specific embodiments comprise a vest and an infant carrier configured to be coupled to the vest. In certain embodiments the infant carrier comprises a rigid shell and a soft insert. In particular embodiments the medical equipment support device comprises one or more ties and/or hook-and-loop fasteners. In some embodiments the medical equipment support device is configured as a cover that can be coupled to the infant support device.

Exemplary embodiments include a method of supporting an infant, where the method comprises: obtaining an apparatus as disclosed herein (including for example an apparatus according to any of the claims); securing an infant in the infant support device; and securing medical equipment with the medical support device.

In the present disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “about” “substantially” or “approximately” mean, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

FIG. 1 illustrates an exemplary embodiment of a support device according to the present disclosure.

FIG. 2 illustrates the embodiment of FIG. 1 during use.

FIG. 3 illustrates an exemplary embodiment of a support device according to the present disclosure.

FIG. 4 illustrates the embodiment of FIG. 3 during use.

FIG. 5 illustrates an exemplary embodiment of a support device according to the present disclosure.

FIG. 6 illustrates an exemplary embodiment of a support device according to the present disclosure.

FIG. 7 illustrates the embodiment of FIG. 6 during use.

FIG. 8 illustrates an exemplary embodiment of a support device according to the present disclosure.

FIG. 9 illustrates the embodiment of FIG. 8 during use.

FIG. 10 illustrates an exemplary embodiment of a support device according to the present disclosure.

FIG. 11 illustrates the embodiment of FIG. 10 during use.

FIG. 12 illustrates an exemplary embodiment of a support device according to the present disclosure.

FIG. 13 illustrates the embodiment of FIG. 12 during use.

FIG. 14 illustrates an exemplary embodiment of a support device according to the present disclosure.

FIG. 15 illustrates the embodiment of FIG. 14 during use.

FIG. 16 illustrates an exemplary embodiment of a support device according to the present disclosure.

FIG. 17 illustrates the embodiment of FIG. 16 during use.

FIG. 18 illustrates an exemplary embodiment of a support device according to the present disclosure.

FIG. 19 illustrates the embodiment of FIG. 18 during use.

FIG. 20 illustrates an exemplary embodiment of a support device according to the present disclosure.

FIG. 21 illustrates the embodiment of FIG. 21 during use.

FIG. 22 illustrates an exemplary embodiment of a support device according to the present disclosure.

FIG. 23 illustrates the embodiment of FIG. 22 during use.

### DETAILED DESCRIPTION OF ILLUSTRATIVE EMBODIMENTS

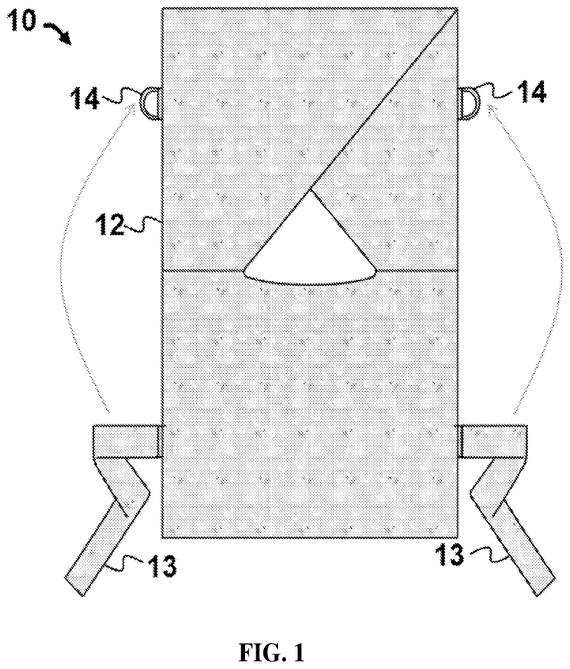

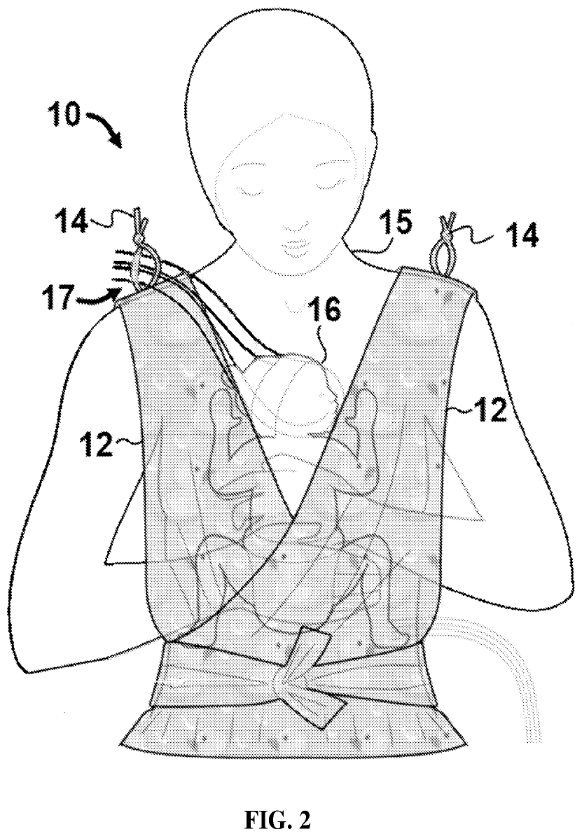

Referring initially to FIGS. 1 and 2, an embodiment of a support apparatus 10 is shown. FIG. 1 illustrates support apparatus 10 before use, while FIG. 2 illustrates device 10 during use. As shown in the figures, in this embodiment support apparatus 10 includes an infant support device 12, as well as one or more medical equipment support devices 14. Medical equipment support device(s) 14 are configured to support, manage and organize medical equipment 17 proximal to an infant 16. For example, support apparatus 10 comprises one or more medical equipment support devices 14 positioned near the shoulder of a user 15 when support apparatus 10 is being worn by user 15. In certain embodiments, medical equipment support device 14 may be configured as one or more ties that can be tied around the medical equipment (e.g. tubes, wires, etc.) associated with infants under medical care to provide support to the medical equipment. In particular embodiments, medical equipment support device 14 may comprise hook-and-loop fasteners (e.g. Velcro®). In the illustrated embodiment, support apparatus 10 further comprises overlapping front panels that open completely to provide easy positioning of infant 16. In this embodiment, support apparatus 10 also comprises fixed loops through which user 15 can pass lower tie straps 13 before positioning support apparatus 10 on his or her boy and supports an infant with apparatus 10. Lower tie straps 13 can then be cinched after infant 16 is positioned to provide support on the bottom and front.

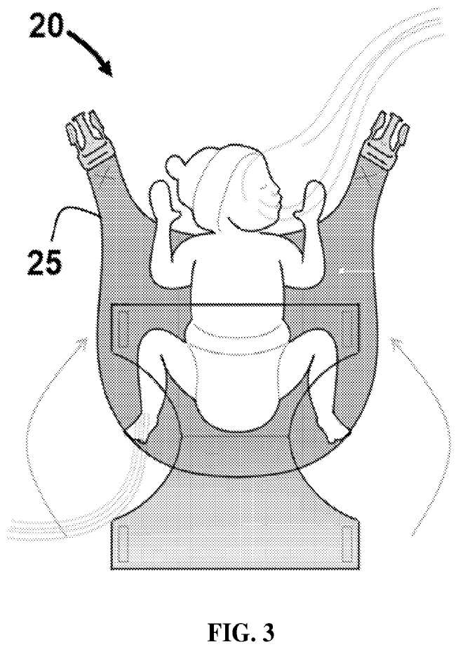

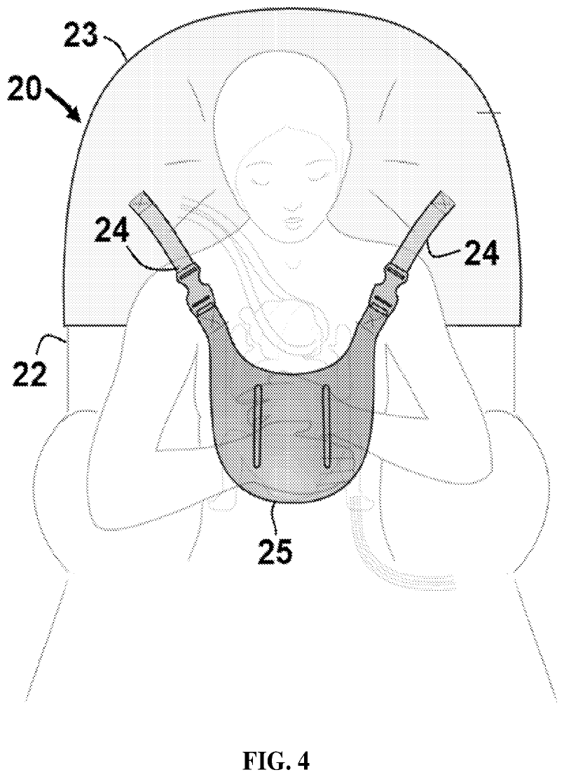

FIGS. 3 and 4 illustrate an embodiment of a support apparatus 20 suitable for use in which a user sits in a chair 22 (including for example, a recliner), which includes a washable cover 23 with adjustable straps 24. The straps on the cover are adjusted for proper baby positioning after the infant is positioned on an infant support device 25 and secured into it. Device 20 can then be buckled to the recliner cover.

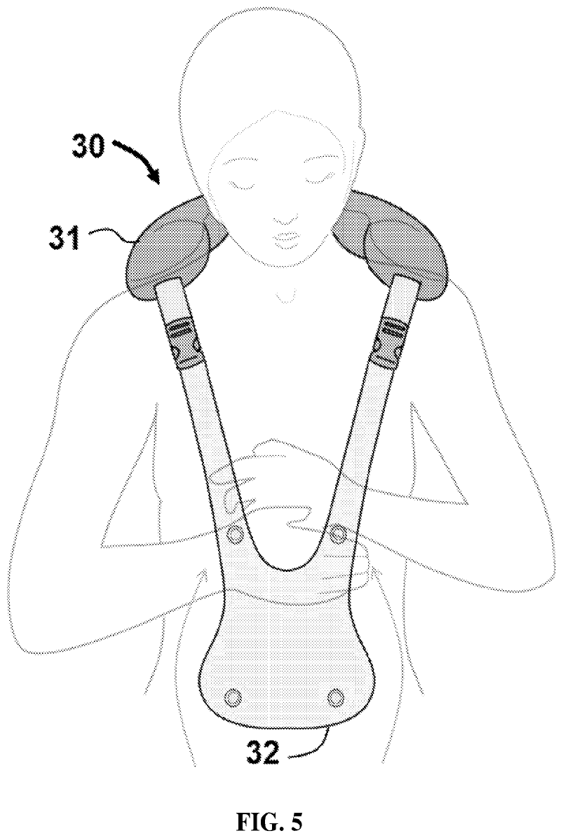

FIG. 5 illustrates an embodiment of a support apparatus 30 that comprises a weighted neck pad 31 similar to a yoke configuration. In certain embodiments, the weighted neck pack may be used with a heated insert for additional comfort to the user. In this embodiment, the user places support apparatus 30 on his or her body and adjusts the straps for proper positioning. The infant can then be secured into a sling 32 configuration formed by support apparatus 30.

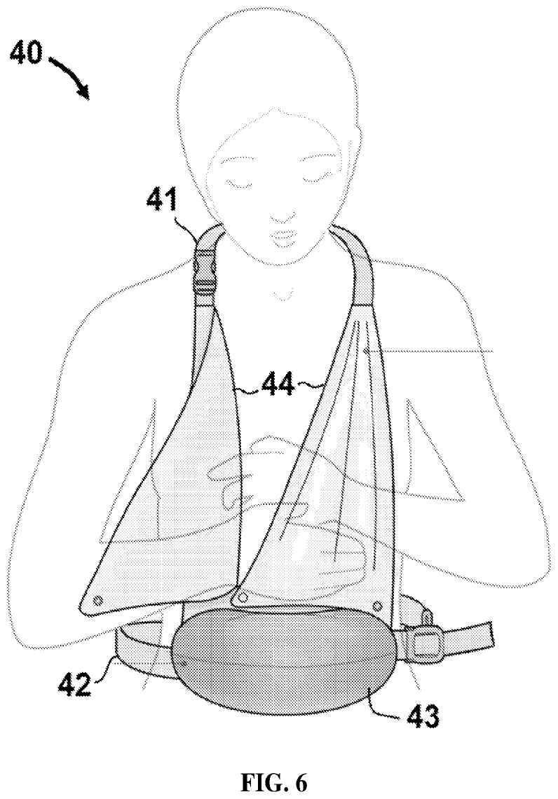

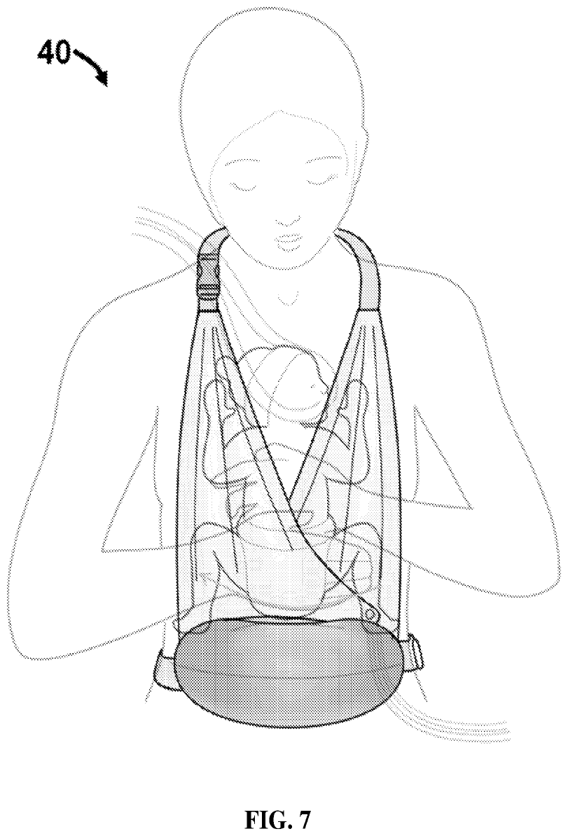

Referring now to FIGS. 6 and 7, an embodiment of a support apparatus 40 is shown during positioning and in use while supporting an infant. This embodiment comprises an adjustable neck strap 41 and adjustable waist strap 42, as well as a cushioned lower support 43 for an infant. As shown in FIG. 6, a user can initially position apparatus 40 on his or her body and adjust waist and neck straps for a proper fit. Apparatus 40 further comprises overlapping panels 44 for a front infant support that snaps to close to the waist strap. The infant can be secured into the apparatus 40 by snapping the front overlapping panels 44 closed.

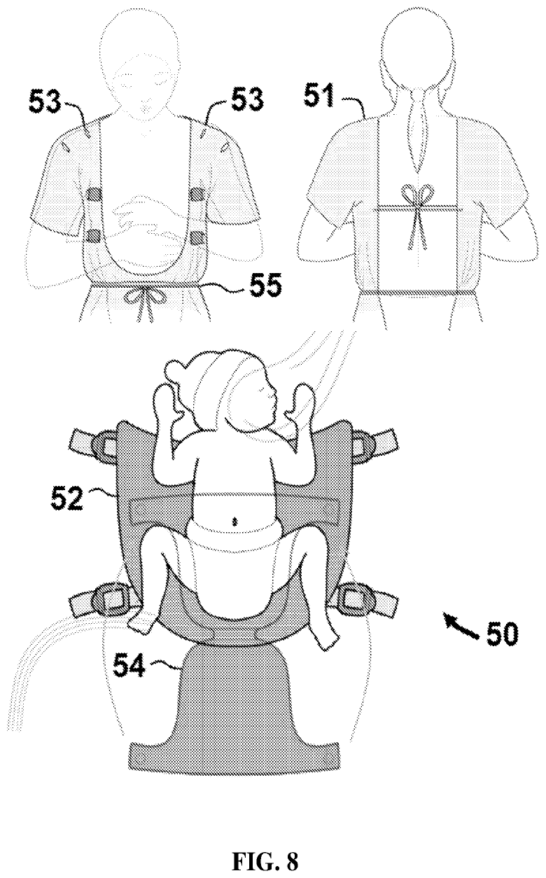

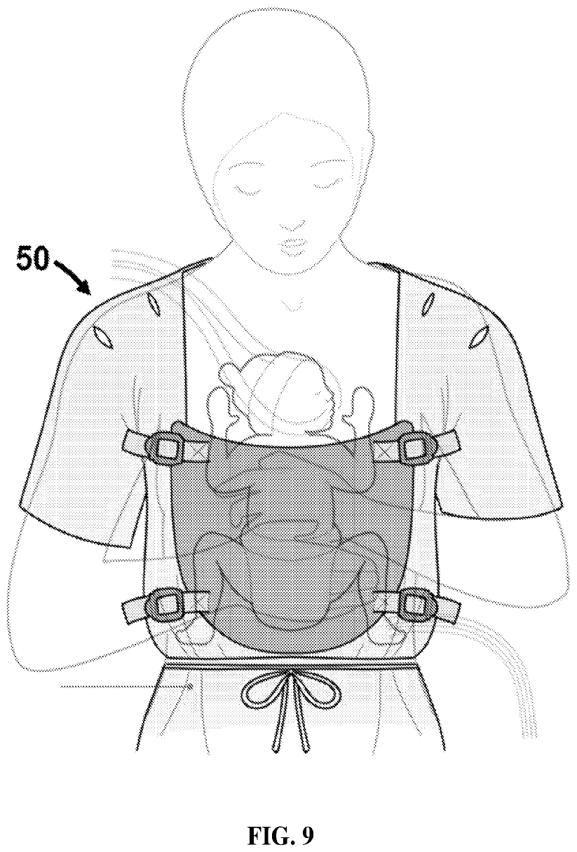

FIGS. 8 and 9 illustrate an embodiment of a support apparatus 50 during initial positioning and in use while supporting an infant. Apparatus 50 comprises a user portion 51 (similar to a shirt or medical gown) and a separate infant support device 52 that can be buckled to user portion 51. This embodiment includes medical device management features 53 that may be configured as slits or cuts in device 50 near the shoulder area of user portion 51 when a user is wearing apparatus 50. As shown in FIG. 8, an infant can initially be placed and secured in the infant support device 52, which includes a lower flap 54 that can be buttoned or snapped to secure which can then be buckled to the user portion. Apparatus 50 further comprises ties 55 that can be tightened or cinched around the user's waist to provide support for the infant.

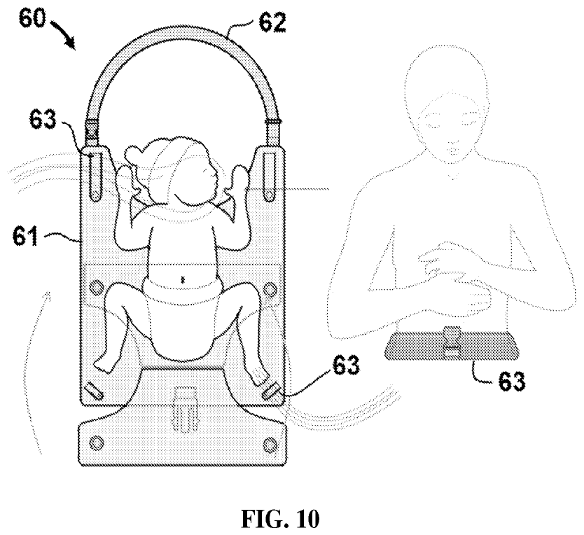

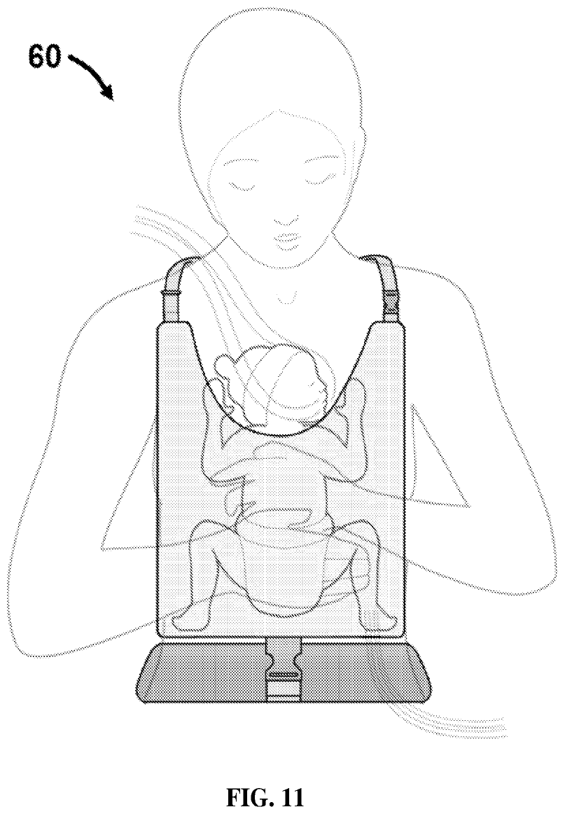

Referring now to FIGS. 10 and 11, an embodiment of a support apparatus 60 is shown during infant positioning and in use while supporting an infant. This embodiment includes an infant support device 61 with a neck strap 62 and a waist band 63. Infant support device 61 comprises medical equipment support device 63 which can be configured as straps that can be placed over the tubes and then coupled to infant support device 61. The infant is initially secured into infant support device 61, which includes a lower flap that can be buttoned or snapped to secure the infant. The neck strap can be adjusted for proper fitment and then placed around the user's neck. The support can then be coupled to the waist band, e.g. via clips or other suitable mechanisms.

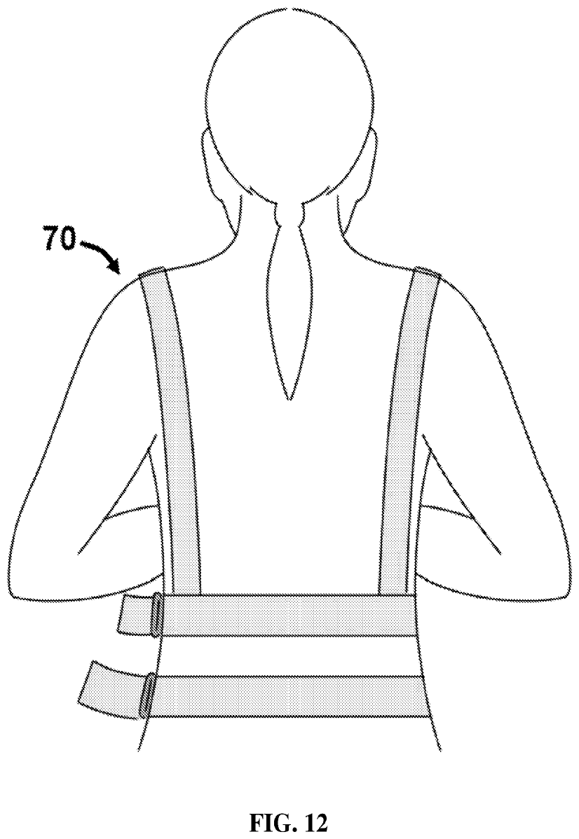

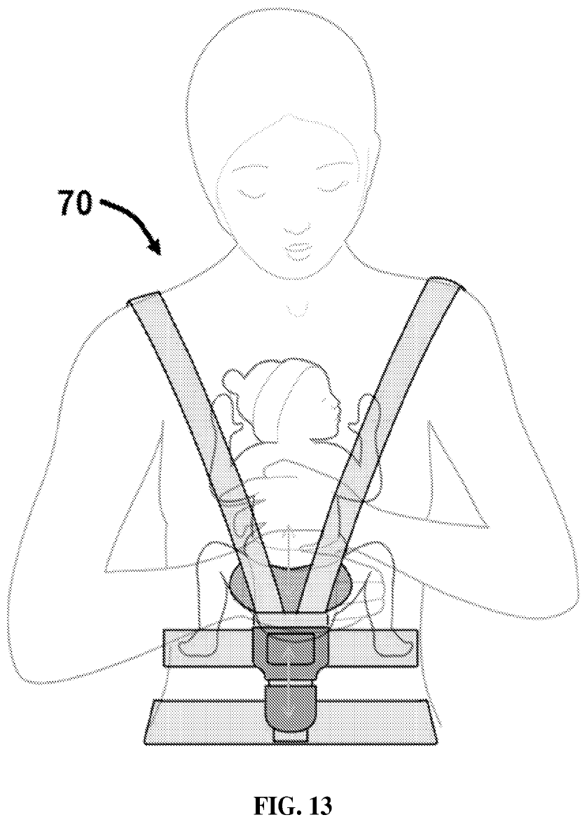

FIGS. 12 and 13 illustrate a support apparatus 70 that comprises a holster configuration. In this embodiment, the user can place apparatus 70 on his or her body and adjust the height of the infant support via straps that extend over the shoulder of the user and form a V-shape in front of the user. The user can also secure apparatus 70 with straps that extend around the waist of the user.

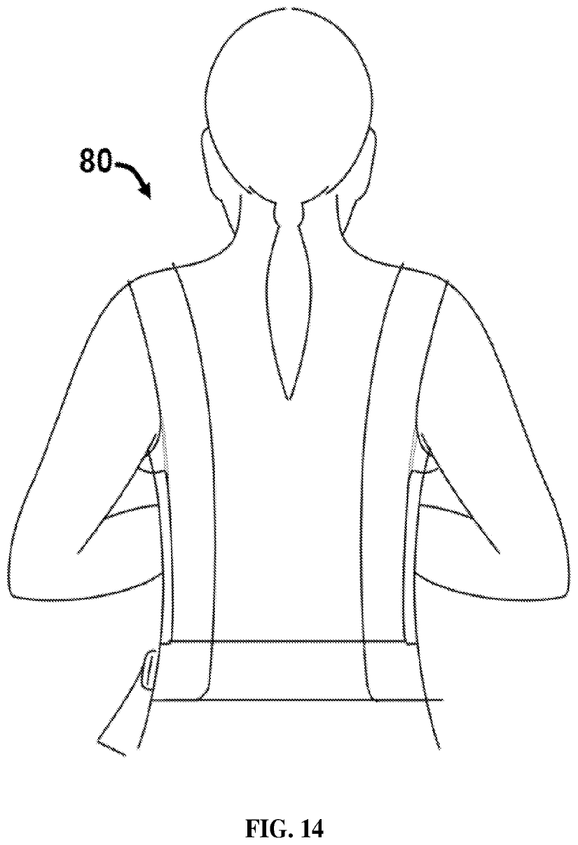

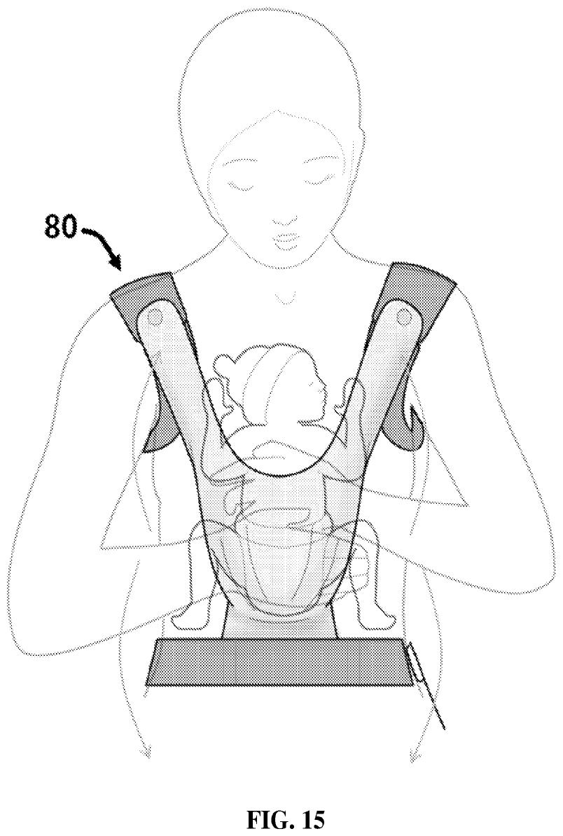

Referring now to FIGS. 14 and 15 a support apparatus 80 that comprises another holster configuration is shown. This embodiment includes straps that extend around the user's shoulder area. In addition, apparatus 80 includes a portion that extends from the shoulder straps to a waist strap. In this embodiment, a user can place apparatus 80 on his or her body and then snap a support flap onto the holster portion (e.g. proximal to the shoulder straps and waist strap). The infant can then be securely placed between the holster and the support flap.

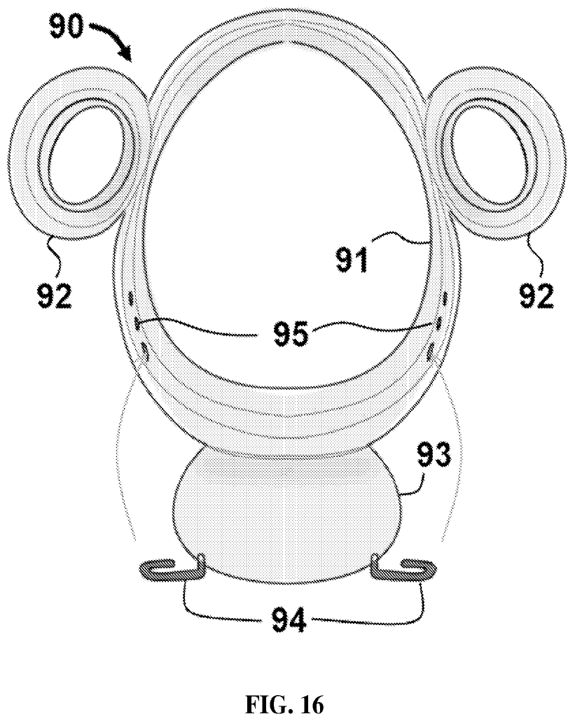

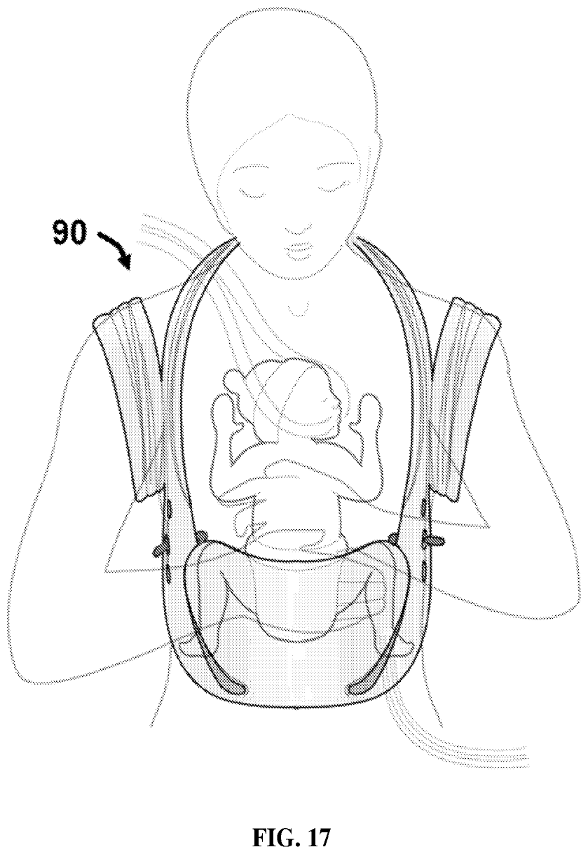

FIGS. 16 and 17 illustrate a support apparatus 90 that comprises a stretchable fabric sling 91. Apparatus 90 further includes armholes 92 through which the user can place his or her arms when wearing the device. In addition, apparatus 90 further comprises a bottom flap 93 coupled to stretchable fabric sling 91. Bottom flap 93 also comprises hooks 94 covered in soft material (e.g. rubber) that can be hooked into holes or slits 95 in sling 91 after the infant is positioned in apparatus 90. The sling can comprise multiple holes or slits to adjust the size for different size infants and provide different locations for positioning the infant with respect to the user.

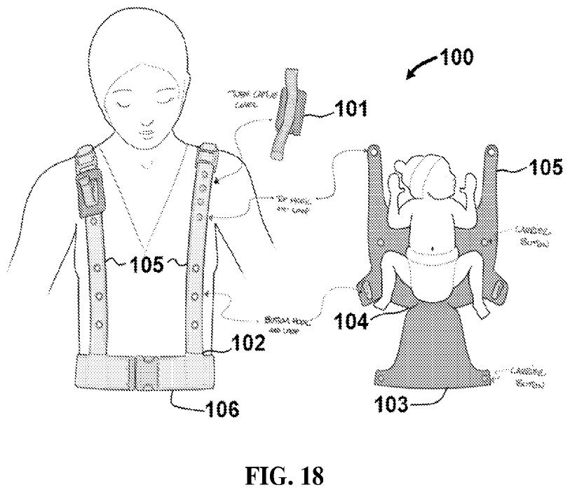

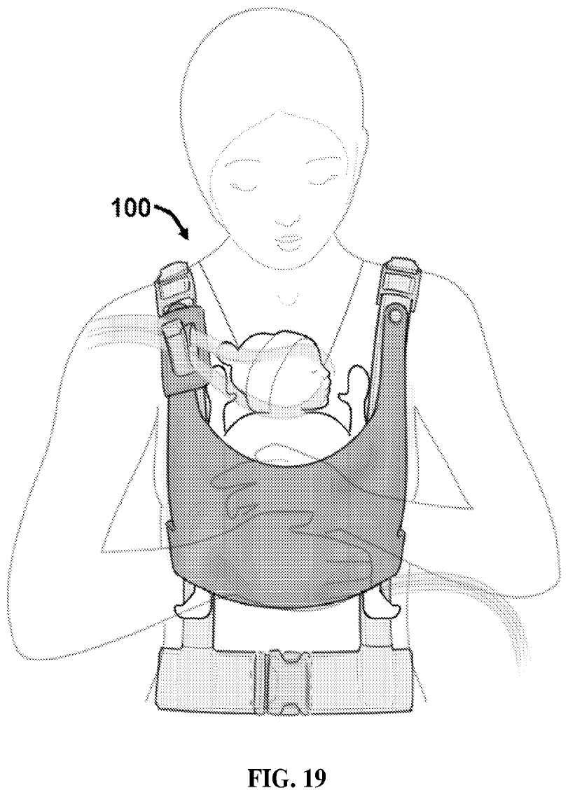

FIGS. 18 and 19 illustrate a support apparatus 100 that comprises a separate support vest 102 that the user wears, and an infant carrier 104 that is coupled to support vest 102. As shown in FIG. 18, the user can place support vest 102 (which comprises upper shoulder straps 105 coupled to a waist belt 106) on his or her body. The infant is secured into the infant carrier, which can include a bottom flap 103 that buttons, snaps or is otherwise secured to an upper portion 105 of the infant carrier. Once the infant is secured in the infant carrier, the infant carrier can be coupled to the support vest as shown by appropriate coupling mechanisms. For example, the support vest and/or infant carrier may comprise a hook and loop configuration, buttons, snaps or other appropriate coupling devices. In addition, support apparatus 100 comprises a medical device retention apparatus that can be coupled to the support vest. In certain embodiments, the medical device retention apparatus is configured as a cover 101 that can be coupled to either side of the upper strap area of apparatus 100.

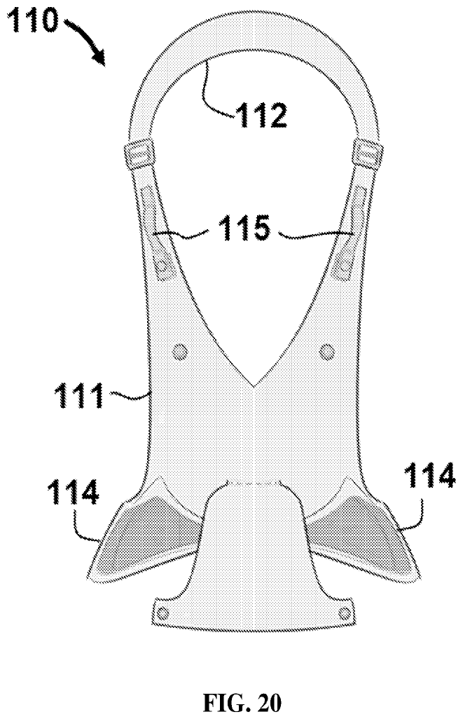

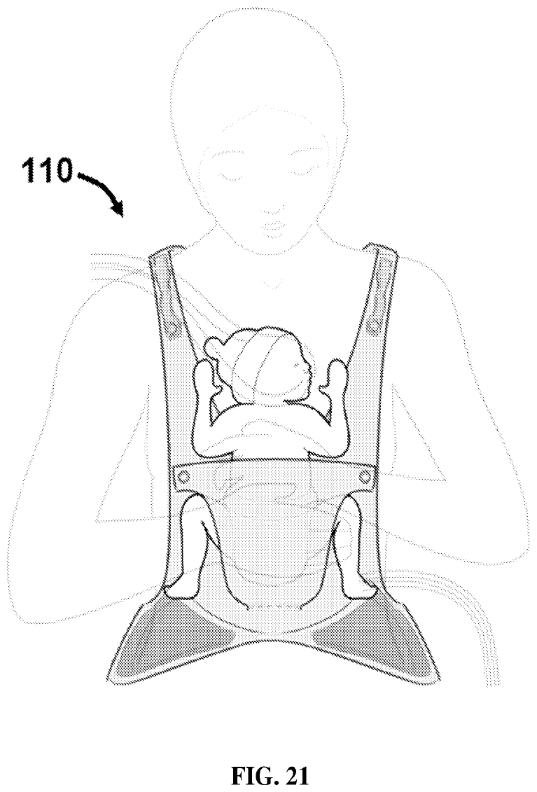

Referring now to FIGS. 20 and 21, a support apparatus 110 is shown comprising a support vest 111 with a strap (or straps) 112 that extend over the user's shoulders and behind the user's neck. The vest includes a flap portion 113 that can be used to secure an infant as shown in FIG. 21. Apparatus 110 further comprises weights 114 in the lower portion of the vest to position the apparatus and infant in the desired location. In the embodiment shown, medical equipment support devices 115 are included on each strap proximal to the user's shoulders. In other embodiments, a medical device management apparatus may be provided on either of the straps near the user's shoulders.

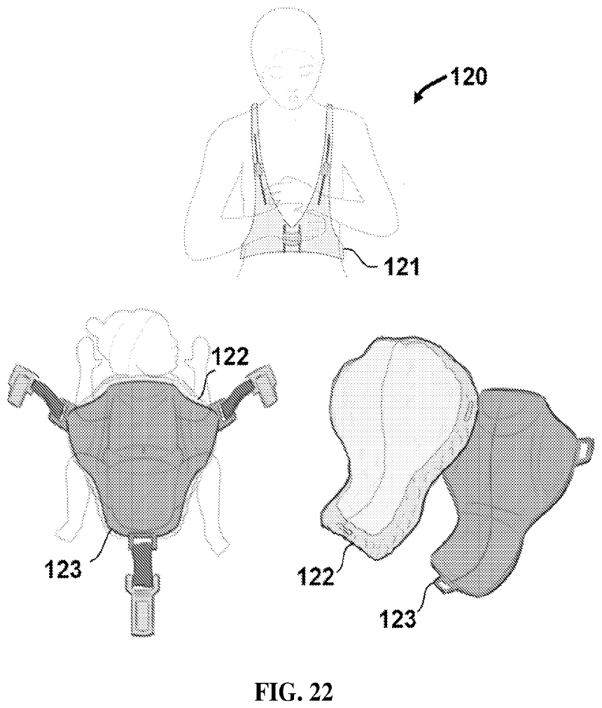

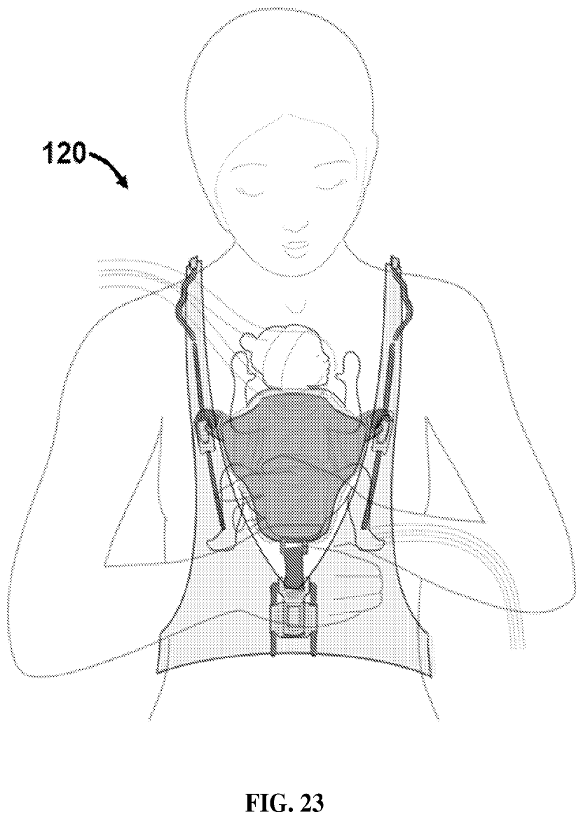

Referring now to FIGS. 22 and 23, a support apparatus 120 comprises a user support vest 121 and a rigid infant carrier shell 122 with a soft insert 123. In FIG. 22, a user is shown wearing the support vest, and an infant is shown placed in the carrier shell with the soft insert between carrier shell and the infant. The carrier shell comprises three straps (two upper straps and one lower strap) that can be coupled to the support vest to support an infant. In the embodiment shown, the carrier shell couples to the support vest via an adjustable rail system that allows for adjustable positioning of the carrier shell. In addition, support apparatus 120 comprises one or more medical device management apparatus proximal to the shoulder area of the user.

As disclosed herein, exemplary embodiments of the present disclosure provide significant benefits and advantages to both patients and care providers.

All of the apparatus, devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:

Feldman et al., Maternal-Preterm Skin-to-Skin Contact Enhances Child Physiologic Organization and Cognitive Control Across the First 10 Years of Life, BIOL PSYCHIATRY 2014; 75:56-64.

Charpak N, Tessier R, Ruiz J G, et al. Twenty-year Follow-up of Kangaroo Mother Care Versus Traditional Care. Pediatrics. 2017; 139(1):e20162063.

1. A support apparatus comprising:
an infant support device configured to support an infant proximal to a user; and
a medical equipment support device configured to support medical equipment proximal to the infant.

2. The support apparatus of claim 1 wherein the infant support device comprises overlapping panels.

3. The support apparatus of claim 1 wherein the infant support device comprises a cover configured to be coupled to a chair.

4. The support apparatus of claim 1 wherein the infant support device comprises a neck pad.

5. The support apparatus of claim 4 wherein the neck pad is heated.

6. The support apparatus of claim 1 wherein the infant support device comprises an adjustable neck strap.

7. The support apparatus of claim 1 wherein the infant support device comprises an adjustable waist strap.

8. The apparatus of claim 1 wherein the infant support device comprises a shirt configuration.

9. The apparatus of claim 8 wherein the medical equipment support device comprises one or more holes in the shirt configuration.

10. The apparatus of claim 1 wherein the infant support device comprises an upper portion and a lower flap portion.

11. The apparatus of claim 10 wherein the lower flap portion can be coupled to the upper portion.

12. The apparatus of claim 11 wherein the lower flap portion can be coupled to the upper portion via buckles.

13. The apparatus of claim 11 wherein the lower flap portion can be coupled to the upper portion via hooks that engage holes in the upper portion.

14. The apparatus of claim 1 wherein the infant support device comprises a holster configuration.

15. The apparatus of claim 1 wherein the infant support device comprises a sling.

16. The apparatus of claim 14 wherein the sling comprises a stretchable fabric.

17. The apparatus of claim 1 wherein the infant support device comprises a vest and an infant carrier configured to be coupled to the vest.

18. The apparatus of claim 17 wherein the infant carrier comprises a rigid shell and a soft insert.

19. The apparatus of claim 1 wherein the medical equipment support device comprises one or more ties.

20. The apparatus of claim 1 wherein the medical equipment support device comprises hook-and-loop fasteners.

21. The apparatus of claim 1 wherein the medical equipment support device is configured as a cover that can be coupled to the infant support device.

22. A method of supporting an infant, the method comprising:
obtaining an apparatus according to any of the preceding claims;
securing an infant in the infant support device; and
securing medical equipment with the medical support device.
